(19) United States
(12) Patent Application Publication
(10) Pub. No.: US 2003/0224464 A1
(43) Pub. Date: Dec. 4, 2003

(54) Secreted caveolin as a marker for prostate cancer

(75) Inventor: Timothy C. Thompson, Houston, TX (US)

Correspondence Address: VINSON & ELKINS, L.L.P., 1001 FANNIN STREET, 2300 FIRST CITY TOWER, HOUSTON, TX, 77002-6760, US

(73) Assignee: Baylor College of Medicine

(21) Appl. No.: 10/355,259

(22) Filed: Jan. 31, 2003

Related U.S. Application Data
(60) Provisional application No. 60/352,513, filed on Jan. 31, 2002.

(51) Int. Cl. G01N 33/574

(52) U.S. Cl. 435/7.23

(57) ABSTRACT

Prostate cancer cells secrete cav-1 and that secreted cav-1 can stimulate viability and clonal growth in prostate cancer cells that do not express cav-1. The concept of a secreted autocrine or paracrine factor that directly contributes to androgen resistance in prostate cancer is novel and represents an efficient mechanism for maximizing resistance to various pro-apoptotic stimuli that metastatic cells often encounter during the highly inefficient process of metastasis. The detection of secreted cav-1 in patient sera has significant potential for clinical utility. Since, unlike PSA, secreted cav-1 is linked to malignant characteristics of prostate cancer cells, serum cav-1 has a unique prognostic and diagnostic capacity. The levels of cav-1 protein within prostate cancer tissues were evaluated directly using immunohistochemistry and RT-PCR as well as serum cav-1 levels in men who undergo radical prostatectomy with lymph node dissection. This identified prostate cancer related cav-1 as a bio-marker with unique clinical potential including independent value in predicting biochemical recurrence following radical prostatectomy and prognostic significance. Cav-1 can also provide clinically useful prognostic information prior to surgery (biopsy and serum cav-1), is useful as a prognostic bio-marker, and also for the ability to predict the recurrence of prostate cancer.

### REFERENCE TO RELATED APPLICATIONS

This application claims priority to U.S. Provisional application No. 60/352,513, entitled “Secreted Caveolin as a marker for Prostate Cancer” filed Jan. 31, 2002.

### RIGHTS IN THE INVENTION

This invention was made with government support under grant number RO1 68814 and program project grant number P50-58204, both awarded by the National Cancer Institute, National Institutes of Health, and the United States government may have certain rights in the invention.

### BACKGROUND

1. Field of the Invention

This invention relates to caveolin as a marker for cancer and metastatic disease and, in particular, to diagnostic and prognostic kits, aids and methods associated with the use of serum caveolin for the detection of prostate cancer.

2. Description of the Background

The precise risk factors for prostate cancer are unknown with both genetic factors and environmental factors likely to be involved. African-American men have a much higher incidence of and mortality from prostate cancer than White-American men. The widespread use of serum PSA-based screening of asymptomatic men resulted in a sharp (over two fold) increase in the detection of incidences of prostate cancer during the 1990s. During this time, the age-specific mortality rate from prostate cancer also increased. Although, the incidence and mortality rates have recently begun to decrease it is unclear if these reductions relate to increased screening and earlier aggressive treatment, misclassification of cause of death, or more complex population dynamics. Also there probably has not been adequate follow-up time to make meaningful comparisons of screening or treatment outcomes. The treatments currently used for presumably localized disease are indeed exclusively local treatments that are designed to ablate the tumor either surgically or with irradiation. The optimal use of these therapies requires accurate staging of the disease. Unfortunately neither low to intermediate serum PSA levels (2-15 ng/ml) or currently available clinical modalities are capable of accurate staging in the low range. Therefore, one possible explanation for the low impact of prostate cancer therapy thus far is that occult metastases were present at the time of treatment in many cases when localized treatment was administered. Metastatic disease present at the time of treatment would invariably continue to progress. The reported failure rate, within 5 years as indicated by rising prostate-specific antigen levels for patients undergoing radical prostatectomy, ranges from 20% to 57%, indicating the presence of either local tumor recurrence and/or occult metastasis.

The pathological assessment of prostate cancer prior to treatment is complicated by its heterogeneous presentation. Although a prominent index cancer is typically present, it has long been recognized that prostate cancer is multifocal, usually contains more than one histological grade, and often is juxtaposed and admixed with other benign pathology such as benign prostatic hyperplasia (BPH). Malignant potential is currently most often assessed by the grading system proposed by Gleason. Yet examination of radical prostatectomy specimens of non-palpable cancers has revealed that up to 45% of high-grade tumors (Gleason grade 4 or 5) were less than 1 cm³ in volume. These clinical data point to the possibility that highly aggressive disease may present early as small tumors and not necessarily evolve in a predictable fashion from low-grade tumors. The results of other studies also indicate that although there is a general relationship between tumor volume and metastatic progression, relatively small tumors that are confined to the prostate may also seed metastases. These clinical observations have been supported by the results of in vivo experiments that indicate metastases do not necessarily originate from the most abundant clone of the malignant cells at the primary site.

Significantly, the screening and treatment efforts of the last decade have resulted in a new and highly complex subset of patients who have experienced recurrence after treatment with these localized therapies. There are now tens of thousands of men who have had recurrence who have rising serum PSA levels indicating either local or distant disease recurrence after radical prostatectomy or irradiation therapy. Additional confounding problems with prostate cancer is that the prevalence of histologic cancers with low malignant potential is high (about 40% in men >50 years old suggesting that an increasing proportion of cancers that have been detected over the last decade, and are currently being detected may in fact be “clinically unimportant.” Data from some studies suggest that 10%-26% of nonpalpable cancers detected by PSA screening are “clinically unimportant” on the basis of pathologic criteria, e.g., less than 0.5 cc, Gleason sum ≦6, and disease confined to the prostate. The detection and treatment of potentially “clinically unimportant cancers” with potentially harmful therapy, such as radical prostatectomy and irradiation therapy, may not be appropriate in many cases. Overall, the complex morphologic patterns, histologic heterogeneity, and the early manifestations of high malignant potential preclude a straightforward assessment of the metastatic potential of localized prostate cancer and indicate the need for additional clinical and pathological tests for the objective assessment of prostate cancer stage and biological/clinical potential.

### SUMMARY OF THE INVENTION

The present invention overcomes the problems and disadvantages associated with current strategies and designs, and provides new tools and methods for the detection of neoplasia and metastatic and associate diseases.

One embodiment of the invention is directed to methods for detecting a neoplasia in a patient comprising determining a level of a caveolin in a biological sample obtained from the patient. The neoplasia may be a malignant or non-malignant cancer including, but not limited to, breast cancer, esophageal cancer, head and neck cancer, liver cancer, lung cancer, gastrointestinal cancer, pancreatic cancer, prostate cancer, skin cancer, stomach cancer, a metastasis, a micrometastasis, and combinations thereof. Suitable biological samples include blood, plasma, serum, tissue, interstitial fluid, and combinations thereof.

Another embodiment of the invention is directed to kits for detecting the level of a caveolin in a biological sample obtained from a patient suspected of having a neoplastic disorder comprising an agent that can be quantitatively detected upon association with said caveolin. Suitable agents for use with the kit include, for example, an anti-caveolin antibody, a caveolin receptor, and functional fragments and combinations thereof.

Another embodiment of the invention is directed to methods for detecting prostate cancer in a patient comprising obtaining a sample of blood from a patient, fractionating said sample into one or more fractions, determining a level of caveolin in at least one fraction, comparing the level of caveolin determined in said fraction with the level of caveolin determined in fractions obtained from similar biological samples obtained from non-cancerous patients, and determining the presence or absence of prostate cancer in said patient.

Another embodiment of the invention is directed to methods for determining a metastatic potential of a primary prostate tumor comprising coupling an anti-caveolin antibody to a detectable marker, contacting a sample of the tumor with the anti-caveolin antibody coupled to the detectable marker, and determining the amount of anti-caveolin antibody bound to the sample.

Another embodiment of the invention is directed to methods for determining a metastatic potential of a primary prostate tumor comprising coupling an anti-caveolin antibody to a detectable marker, contacting a sample of the tumor with the anti-caveolin antibody coupled to the detectable marker, and determining the amount of anti-caveolin antibody bound to the sample.

Another embodiment of the invention is directed to reagents for determining the metastatic potential of a primary prostate tumor comprising an anti-caveolin antibody coupled to a detectable marker.

Another embodiment of the invention is directed to methods for detecting prostate cancer, comprising extracting a serum sample from a patient, separating the serum sample into lipid fractions, contacting the HDL₃ fraction of serum sample with an anti-caveolin antibody coupled to a detectable marker, measuring the amount of anti-caveolin antibody bound to the HDL₃ fraction of the serum sample, and determining the caveolin-1 concentration in the serum sample.

Other objects and advantages of the invention are set forth, in part, in the description, which follows, and in part, will be obvious from this description and may be learned from the practice of the invention.

### DESCRIPTION OF THE FIGURES

FIG. 1 Frequency of cav-1 positive specimens in normal prostate, prostate cancer primary tumors and metastases in patients with or without hormonal therapy.

FIG. 2 Suppression of in vivo metastatis activities in antisense cav-1 clones.

FIG. 3 Cav-1 doses-dependent cell protection from thapsigargin-induced cell death in LNCaP cells.

FIG. 4 Inhibition of caspase activation by cav-1 in LNCaP cells.

FIG. 5A Western blot of LNCaP lysates infected with adenoviral vectors or transfected with plasmids.

FIG. 5(B) Kinase activity assay after PDK1 IP: Infected or transfected LNCaP cell lysates precipitated with IgG or anti-PDK1 then used for an in vitro kinase assay.

FIG. 6(A) Kinase activity in cell lysates after IP with Akt Ab: Purified protein (Akt, GSK-3, or Bad) was used as kinase substrate then detected with P-specific Ab.

FIG. 6(B) Phosphorylation of purified Bad protein by cell lysate followed by Western blot with P-specific Ab.

FIG. 7 Effect of PI-3K inhibitors on cav-1 mediated Akt phosphorylation.

FIG. 8 Cav-1 stabilizes Akt phosphoprotein levels.

FIG. 9 Secretion of cav-1 from cell lines.

FIG. 10 Secreted cav-1 from HP-LNCaP cells increase viability and survival.

FIG. 11 Cav-1 antisera suppresses 178-2 BMA orthotopic tumor growth and metastasis.

FIG. 12 Detection of cav-1 in HDL3 fraction of serum.

FIG. 13 Cav-1 standard curves sandwich ELISA at 5-day intervals.

FIG. 14 Serum cav-1 in control and prostate cancer patients.

### DESCRIPTION OF THE INVENTION

As embodies and broadly described herein, the present invention is directed to tools and methods for the diagnosis of neoplasia and related metastatic and associated diseases, and in particular, prostate cancer.

Insight into the progression of prostate cancer has been developed through investigations of caveolin-1 (cav-1) expression and specifically in metastatic disease. Clinical studies in this area have followed a logical progression from the identification of cav-1 overexpression in metastatic prostate cancer (G. Yang et al., Clin. Cancer Res. 4:1873-80, 1998); the determination of cav-1 as an independent prognostic marker for prostate cancer progression in lymph node negative patients who have recurred following radical prostatectomy (G. Yang et al., Cancer Res. 59:5719-23, 1999), and a significant association of increased cav-1 in prostate cancer in African-American men vs. White-American men ( )G. Yang et al., Clin Cancer Res. 6:3430-33, 2000). Research studies have elucidated one aspect of the mechanism of action of cav-1 by showing that cav-1 has anti-apoptotic properties under a variety of clinically relevant circumstances including growth factor deprivation and oncogene overexpression. In addition, these studies contributed to an understanding of androgen-insensitive prostate cancer. In previous studies, the aberrant expression of HER-2/neu has been implicated in androgen independence in animal models and by immunohistochemical analyses of human specimens. However, the role of HER-2/neu in prostate cancer progression is not as self-evident as it is in breast cancer. Previous studies have also documented that bcl-2 overexpression may characterize a subset of androgen-insensitive disease. Recently, it was demonstrated that cav-1 upregulation is associated with the development of androgen-insensitive prostate cancer and that androgen-insensitive prostate cancer cells secrete biologically active cav-1 in a steroid-regulated fashion. Testosterone (T) upregulates cav-1 expression in prostate cancer cells in part through transcriptional activation. Therefore, in the presence of T, cav-1 expression and/or secretion may be significantly stimulated in prostate cancer cells. Androgen ablation may select for alternative pathways of cav-1 regulation. It was previously shown that polypeptide growth factors can regulate cav-1 expression in NIH 3T3 cells. A variety of relevant polypeptide growth factors including FGF-2 and TGF-β1 can stimulate cav-1 expression. Therefore cav-1 expression and secretion may be stimulated initially by androgens, yet subsequent androgen ablation may select for alternative pathways that sustain cav-1 activities and thus transition the malignant cell into an androgen-insensitive phenotype.

It has been surprisingly discovered that prostate cancer cells secrete cav-1 and that secreted cav-1 can stimulate viability and clonal growth in prostate cancer cells that do not express cav-1 (S. A. Tahir et al., Cancer Res. 61:3882-85, 2001). The concept of a secreted autocrine or paracrine factor that directly contributes to androgen resistance in prostate cancer is novel and represents an efficient mechanism for maximizing resistance to various pro-apoptotic stimuli that metastatic cells often encounter during the highly inefficient process of metastasis. Although other cells types produce cav-1 the secretion of cav-1 appears to be much more limited. Of the two reports on secreted cav-1, one study demonstrated secretion of cav-1 by normal pancreatic exocrine cells and the other documented secretion of cav-1 by prostate cancer cells. The predicted biological activities of secreted cav-1 indicate that this molecule represents a potential therapeutic target for prostate cancer. Proof of principle for this concept has been established in that it is shown that cav-1 antibody can have anti-metastatic activities in vivo when administered systemically and cav-1 is present in the serum of prostate cancer patients.

The detection of secreted cav-1 in patient sera has significant potential for clinical utility. Since, unlike PSA, secreted cav-1 is linked to malignant characteristics of prostate cancer cells, serum cav-1 may have unique prognostic and/or diagnostic capacity. Previous work laid a strong foundation upon which to pursue the clinical utility of prostate cancer-derived cav-1. The levels of cav-1 protein within prostate cancer tissues can be evaluated directly using immunohistochemistry and RT-PCR as well as serum cav-1 levels in men who undergo radical prostatectomy with lymph node dissection. Other biochemical bio-markers that have a potential relationship to cav-1 can be assessed and a comprehensive correlation analysis among these variables determined. This provides a clear picture of the clinical profile, interrelationships and associations of prostate cancer related cav-1, other related bio-markers and specific clinical and pathological variables. In addition, it identifies bio-marker combinations that may have unique clinical potential. On the basis of radical prostatectomy specimens from lymph node negative patients that demonstrated tissue cav-1 has independent value in predicting biochemical recurrence following radical prostatectomy, a retrospective study to determine the prognostic significance of tissue cav-1 (needle biopsy and radical prostatectomy specimens) as well as serum cav-1 levels in a well-defined patient population with long term follow-up can be determined. This can determine the capacity of cav-1 to provide clinically useful prognostic information prior to surgery (biopsy and serum cav-1) and evaluate the capacity of serum cav-1, in general, to serve as a prognostic bio-marker. Tissue (needle biopsy and radical prostatectomy) and serum cav-1 can also be evaluated for their ability to predict biochemical recurrence in a prospective trial in men who undergo radical prostatectomy and plasma cav-1 can be ascertained as a tool for the identification of men at risk for the development of prostate cancer.

One embodiment of the invention is directed to a method for detecting a neoplasia in a patient comprising determining a level of a caveolin in a biological sample obtained from the patient. The neoplasia may be any neoplastic disorder such as, but not limited to, a neoplasia such as breast cancer, esophageal cancer, head and neck cancer, liver cancer, lung cancer, gastrointestinal cancer, pancreatic cancer, prostate cancer, skin cancer, stomach cancer, a metastasis, a micrometastasis, and combinations thereof. Suitable biological samples include most any fluid or tissue of the body such as, for example, blood, plasma, serum, tissue, interstitial fluid, and combinations thereof. Preferably, the cancer is prostate cancer, and the caveolin is cav-1. The biological sample may be serum that has been fractionated into different portions that contain caveolin associated with lipoproteins. The level of caveolin associated with the same or unique lipoproteins indicates the presence or absence of disease. By comparing the levels determined with each other or with known levels in similar biological samples from patients that do not have a neoplastic disorder, can indicate the presence or absence of disease.

In another embodiment, the invention includes a kit for practicing the above methods. Kits detect the level of a caveolin in a biological sample obtained from a patient suspected of having a neoplastic disorder and can comprise an agent that can be quantitatively detected upon association with said caveolin. Suitable agents for use with the kit may include, but are not limited to, one or more of an anti-caveolin antibody, a caveolin receptor, and functional fragments and combinations thereof.

In another embodiment, the invention includes a method for detecting prostate cancer in a patient comprising obtaining a sample of blood from a patient, fractionating said sample into one or more fractions, determining a level of caveolin in at least one fraction, comparing the level of caveolin determined in said fraction with the level of caveolin determined in fractions obtained from similar biological samples obtained from non-cancerous patients, and determining the presence or absence of prostate cancer in said patient.

In another embodiment, the invention includes a method for determining a metastatic potential of a primary prostate tumor comprising coupling an anti-caveolin antibody to a detectable marker, contacting a sample of the tumor with the anti-caveolin antibody coupled to the detectable marker, and determining the amount of anti-caveolin antibody bound to the sample.

In another embodiment, the invention includes a method for determining a metastatic potential of a primary prostate tumor comprising coupling an anti-caveolin antibody to a detectable marker, contacting a sample of the tumor with the anti-caveolin antibody coupled to the detectable marker, and determining the amount of anti-caveolin antibody bound to the sample.

In another embodiment, the invention includes a reagent for determining the metastatic potential of a primary prostate tumor comprising an anti-caveolin antibody coupled to a detectable marker.

In another embodiment, the invention includes a method for detecting prostate cancer, comprising extracting a serum sample from a patient, separating the serum sample into lipid fractions, contacting the HDL₃ fraction of serum sample with an anti-caveolin antibody coupled to a detectable marker, measuring the amount of anti-caveolin antibody bound to the HDL₃ fraction of the serum sample, and determining the caveolin-1 concentration in the serum sample.

The following examples are offered to illustrate embodiments of the invention are should not be viewed as limiting the scope of the invention.

### EXAMPLES

Cav-1 is Overexpressed in Virulent Prostate Cancer

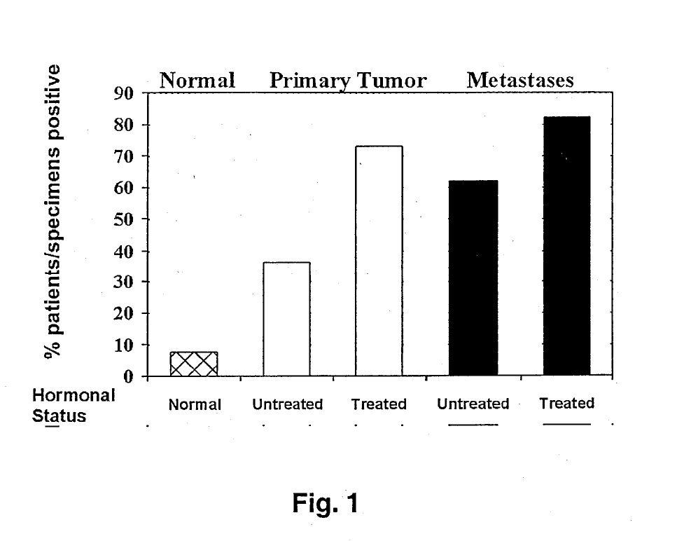

Cav-1 expression was comprehensively examined in prostate cancer tissues by immuno-histochemistry. Normal prostate tissues was evaluated from either cadaveric organ donor prostates or from men without prostate cancer who underwent a cystoprostatecomy as well as a large panel of primary tumor tissues and specimens of lymph node from prostate cancer patients who had undergone a radical prostatectomy and stage D prostate cancer patients undergoing hormonal treatment (FIG. 1). Published studies using this set of tissue specimens demonstrated that the frequency of cav-1 positivity was 8% in normal glandular epithelia; 14% in pathologically localized prostate cancer; 38% in primary cancers with nodal metastases; and 62% in the nodal metastases per sé (20, 30). In this series, the frequency of cav-1 positive primary prostate cancers increased from 38% in the hormonally naive patient group to 73% in the hormone refractory patient group (p<0.05, χ²test). Cav-1 positivity increased from 62% in metastatic specimens from patients who had not been treated with hormone therapy to 82% of metastases from patients treated with hormones (P<0.05, Mann-Whitney test). The percentage of cav-1 positive cells was significantly increased from 18.6% in untreated primary tumors to 29.9% in hormone-treated primary tumors (P<0.05, Mann-Whitney test). The percentage of cav-1 positive cells was also increased in metastatic specimens of untreated patients from 35.5% to 38% in specimens from treated patients but this increase was not significant (P>0.05, Mann-Whitney test). Increased cav-1 positivity in hormone refractory prostate cancer is consistent with several reports that have correlated overexpression of cav-1 with multidrug resistance independent of P-glycoprotein in human cancer cell lines from various tumor types. Consistent with an important functional activity, cav-1 overexpression in primary tumors from lymph node negative patients is an independent predictor of recurrence following radical prostatectomy. These studies exclusively involved immunohistochemical staining analysis of either radical prostatectomy or autopsy specimens. This type of analysis is clinically useful for the determination of cav-1 positivity by immunohistochemistry in pretreatment biopsies and in serum at various stages during the course of the disease, particularly following treatment, and provides for the clinical utility of cav-1 as a bio-marker. This provides the fundamental data for use of tissue and/or serum cav-1 as a clinical bio-marker for prostate cancer.

Cav-1 is an Anti-apoptotic Gene

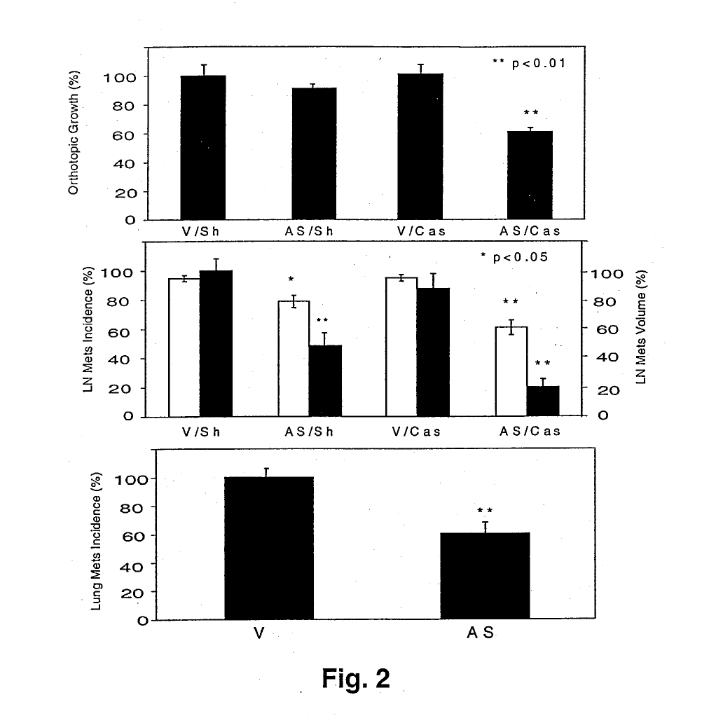

In regard to function, recent data indicate that cav-1 expression suppresses apoptotic cell death in the absence of T in androgen-sensitive mouse prostate cancer cells in vitro and in vivo and that androgen ablation can lead to selection for the outgrowth of cav-1 positive androgen-insensitive mouse prostate cancer cells in vivo. Notably, these experimental results preceded and closely correspond to observations of the occurrence and clinical significance of cav-1 expression in human prostate cancer specimens. These results raised the question of whether T levels can regulate the expression of cav-1. Since normal luminal prostate epithelial cells (the likely precursors of malignant cells) have very low to non-detectable cav-1 levels and a significant fraction of lymph node metastases were shown to express cav-1 in both human and mouse prostate cancer, it seems likely that prostate cancer cells acquire the capacity to upregulate cav-1 during the process of metastatic progression beginning in focal areas in the primary tumor. However, surgical castration also leads to the further selection and outgrowth of cav-1 positive androgen insensitive mouse prostate cancer cells suggesting more complex regulation of cav-1 expression in vivo. Recently, it was shown that T is able to induce cav-1 gene expression at the level of transcriptional activation through AR dependent mechanism(s). In addition, antisense cav-1 was able to significantly inhibit the survival effects of T, indicating that cav-1 is a downstream effector of T-mediated survival activities. To test the effects of cav-1 expression on metastatic activities in vivo, spontaneous (lymph node metastasis from orthotopic tumors) and experimental (tail vein injected cells) metastasis in a panel of high cav-1 lung metastasis-derived mouse prostate cancer cell lines stably transfected with antisense cav-1 (AS) or control vector (V) were analyzed. The growth of the cell lines as orthotopic tumors was compared to vector controls following castration (Cas) or sham (Sh) surgery (FIG. 2A). The AS clones were about 10% smaller than the V clones in the sham operated animals, but this was not a significant difference (P=0.226). However, a significant (39%, P<0.001) decrease in tumor weight was observed in the AS/cas but not in the V/cas. In these same animals the extent of spontaneous lymph node metastasis was evaluated in terms of the number of animals with metastases (incidence) and the relative volume of the metastases as determined by computer assisted microscopic quantitation (FIG. 2B). The AS/Sh clones had less metastatic activity than the vector control clones in sham operated animals with a 17% decrease in incidence (P=0.003) and a 52% reduction in relative volume (P<0.0001). In castrated animals there was no difference in the V clones; however, the AS/Cas clones had a significantly greater decrease in both incidence and volume of lymph node metastasis than AS/Sh (18% and 28% respectively P<0.001). To further evaluate metastatic activity we injected cell clones directly into the tail vein and counted the number of lung metastatic deposits that formed at two weeks (FIG. 2C). The AS cav-1 clones had 40% fewer lung metastases than V control clones (P<0.001).

Overall, these data indicate that T may have a role in stimulating cav-1 expression prior to hormone therapy; that prostate cancer cells acquire the ability to upregulate cav-1 independent of T; and that cav-1 overexpression can lead to increased metastatic activities. Recent and relevant studies have demonstrated that specific kinases in discrete signal transduction pathways (e.g., MAP kinase) can generate activated AR in the absence of T. It has been shown in the prostate cancer cell line LNCaP that increased MAP kinase (erk1/erk2) phosphorylation occurs under serum-free conditions and in the absence of T. Signal transduction pathways involving specific growth factors such as EGF have also been shown to induce the ras-raf1-MAP kinase pathway in prostate cancer cells leading to ligand-independent AR activation. Cav-1 itself has been shown to be involved in the activation of the ras-raf1-MAP kinase pathway through the EGF pathway. This indicates that cav-1 itself could potentially facilitate growth factor-mediated, ligand-independent AR activation through the modulation of specific receptor stimulated ras-raf1-MAP kinase pathway activities. Specific growth factors including PDGF, EGF, FGF-1, FGF-2, VEGF and TGF-β1 can significantly stimulate the expression of cav-1 in the absence of androgens. Therefore it is conceivable that specific growth factors lead to the induction of cav-1, which in turn facilitates ras-raf1-MAP kinase activities that can promote survival and/or proliferation under some conditions. Within the context of prostate cancer this can lead to ligand-independent androgen-receptor-mediated gene expression.

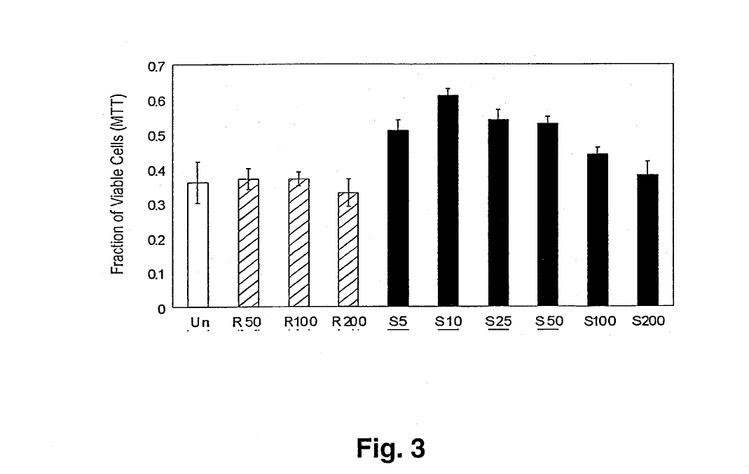

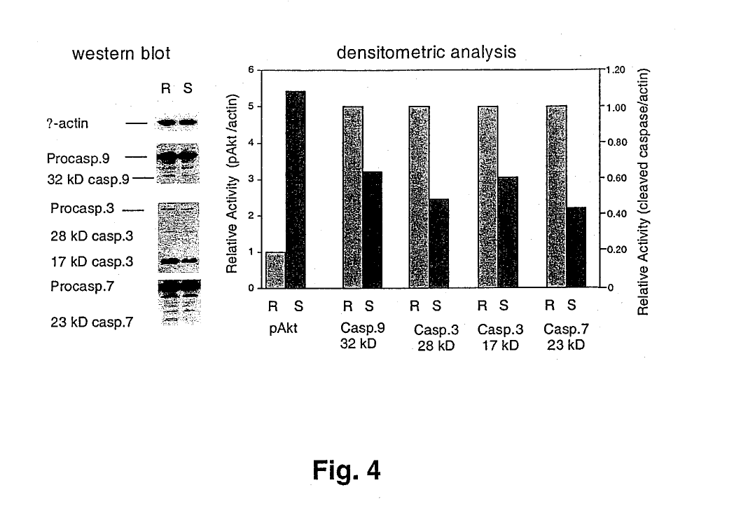

To test for the functional significance of cav-1 overexpression in prostate cancer progression, the possibility that similar to T, cav-1 could suppress apoptosis was considered. For these studies it was necessary to develop models to study apoptosis resistance. Thapsigargin (Tg) a potent endoplasmic reticulum Ca⁺⁺ transporter antagonist leads to BAD dephosphorylation prior to inducing apoptosis in prostate cancer cells. This activity was confirmed in an in vitro model of apoptosis in LNCaP cells. Using this model it was demonstrated that overexpression of cav-1 can protect cells from Tg-induced apoptosis leading to enhanced survival (FIG. 3). To evaluate the molecular effectors of apoptosis that are inhibited by cav-1 in association with suppression of apoptosis, extensive western blotting studies were performed that demonstrate suppression of caspase 3, 7 and 9 activation (FIG. 4).

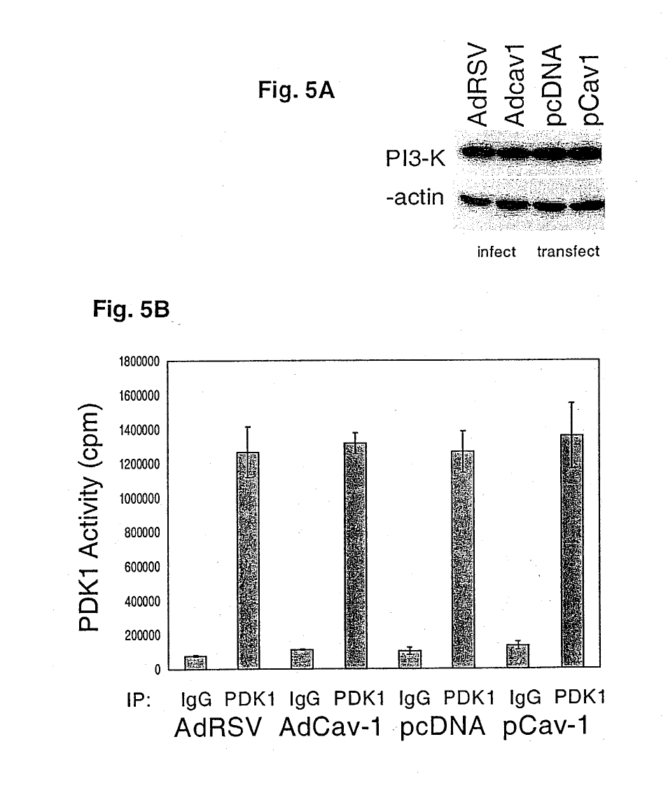

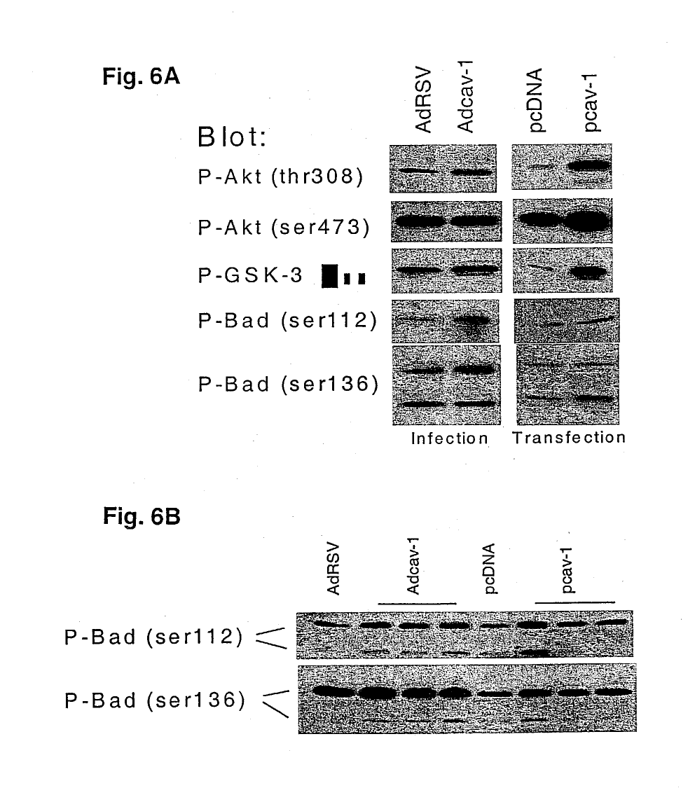

Since the PI3-K/Akt pathway plays an important role in cell survival and cancer progression, including prostate cancer cells, the levels of PI-3K protein and its activity as measured by a the ability to phosphorylate the downstream substrate PDK1 were evaluated to explore potential roles of this pathway in cav-1-mediated cell protection. The results showed that cav-1 overexpression did not affect P13-K levels or PDK1 activity (FIG. 5). Proteins downstream of P13-K and PDK1 were analyzed to test for the mediator(s) of cav-1-mediated cell survival activities. Akt immunoprecipitation kinase assays were performed using either GSK-3 α/β fusion protein or Bad fusion protein as substrates in kinase reactions, followed by western analysis with antibodies specific to phospho-GSK-3 α/β and to phospho-Bad (FIG. 6). The results demonstrated a significantly higher Akt kinase activity in cav-1 expressing LNCaP cells mediated by adenoviral or plasmid vectors than in control cells (FIG. 6A). In an alternative assay using Bad-agarose incubated directly with cav-1 expressing cell lysates, higher levels of phospho-Bad were also observed (FIG. 6B). In parallel, with suppression of apoptosis in LNCaP cells, a suppression of Bad dephosphorylation and increased phosphorylation of Akt and GSK-3 α/β was observed following Tg treatment in LNCaP cells with various levels of cav-1 expression achieved with different moi's of adenoviral vector with cav-1 (S) compared to control adenoviral vector infected cells (R) and transfected cells (FIG. 6A).

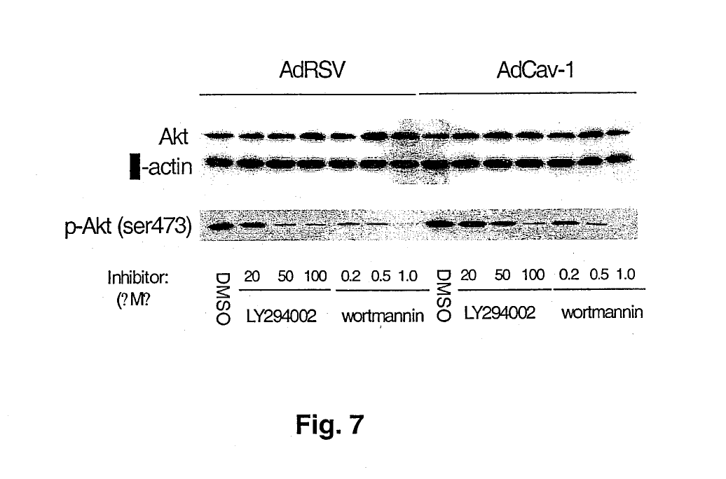

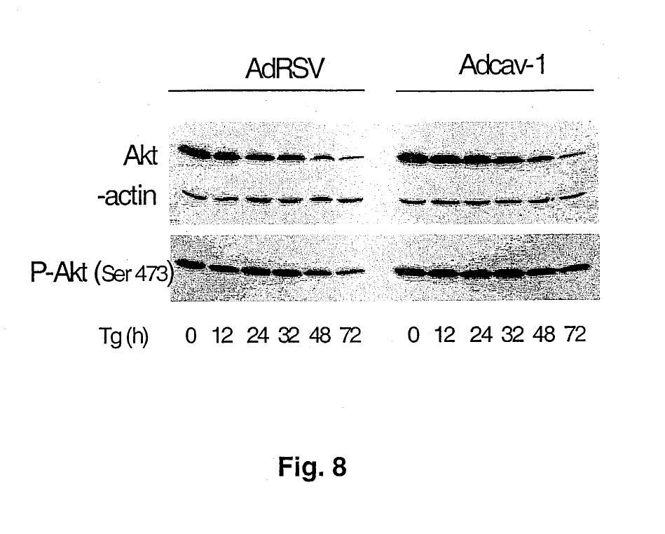

This pathway was assayed using P13-K pathway specific inhibitors LY294002 and wortmannin. FIG. 7 shows that both inhibitors effectively block phosphorylation of Akt, while overall Akt expression is nearly unchanged. Remarkably higher phospho-Akt levels were maintained in cav-1 expressing cells when cells were treated with 20-50 μM LY294002 or 0.2 μM wortmannin. The levels of phospho-Akt were maintained in AdCav-1-infected relative AdRSV-infected cells following Tg treatment over a three day period (FIG. 8). Under these conditions extensive cell death occurs and protection against apoptotic activity was promoted by increased cav-1 (see FIG. 3). As cav-1 has been associated with ras activities and ras can regulate P13-K, which in turn can activate PKB/AKT through phosphorylation, it's conceivable that the cav-1-ras-PI3-K pathway is important in the survival activities stimulated by cav-1 in this model. Overall the data are consistent with a mechanism of action for cav-1 anti-apoptotic activities that include the maintenance of phosphorylated Akt under conditions of cav-1 overexpression. This could be accomplished by either direct interaction of cav-1 with Akt and/or inhibition of specific phosphatase activities. Maintenance of phosphorylated Akt and its activated state then in turn leads to inactivation of GSK-3 α/β and Bad. Additional studies have shown that cav-1 overexpression can also lead to suppression of p38 activities through the activated Akt pathway.

The data demonstrate that cav-1 is a survival factor in prostate cancer and this misdirected function of cav-1 contributes to androgen insensitivity and metastatic activity in prostate cancer. Other documented survival factors overexpressed in prostate cancer include bcl-2 and survivin. Because of the frequent and persistent cav-1 overexpression throughout progression to androgen resistance, cav-1 is a highly critical anti-apoptotic gene in prostate cancer that affects multiple pathways as a result of its unique properties and intracellular localization. In this regard, it is worth comparing cav-1 to the bcl-2 gene family. Bcl-2 and other family members not only manifest many anti-apoptotic functions, but can also block entry into the cell cycle and thus inhibit growth. These activities are documented in the myelomonocyte progenitor cell system where bcl-2 can potentiate cell cycle arrest and the irreversible withdrawal into the nonproliferating (G0) state. Further studies also demonstrate that unlike low levels of bcl-2 protein, which exhibit anti-apoptotic activities, high levels of bcl-2 can exhibit pro-apoptotic activities in human glioma cells. These observations are similar to those made through overexpression of cav-1 in human prostate cancer cells where low to moderate levels of induction of the protein led to pronounced survival activities yet high levels of cav-1 can induce apoptosis. These observations may help to reconcile the contention of some investigators that cav-1 is a tumor suppressor gene based on growth suppressive activities in a limited set of specific cell lines. Although there have been highly selected reports of loss of heterozygosity on human chromosome 7q31.1, the location of the cav-1 gene, in multiple tumor types including prostate cancer, more extensive studies argue against a proper role of cav-1 as a tumor suppressor function. In most studies the 7q31.1 region is as likely to be amplified as lost in prostate cancer, and extensive LOH and mutation analysis specifically of the cav-1 gene have not identified genetic alterations consistent with tumor suppressor activity. Therefore, although cav-1 can suppress growth under some conditions, it does not function as a strictly defined tumor suppressor gene in prostate cancer. Recent studies have shown that overexpression of cav-1 occurs in advanced prostate cancers, high grade bladder cancer, metastatic colon cancer, and esophogeal squamous cell carcinoma and that cav-1 is associated with a drug-resistant phenotype. A multiple-drug-resistant human colon carcinoma cell line and an adriamycin-resistant human breast cancer cell line demonstrated significant cav-1 upregulation independent of P-glycoprotein expression. Independently, significant cav-1 upregulation was also reported in taxol- and epithilone B-resistant lung carcinoma cell lines, and a vinblastine-resistant ovarian cancer cell line. In colon cancer cell lines with low basal levels of cav-1, cell survival by selection for either drug resistance or increased metastatic potential correlated with increased cav-1 expression levels. These data linking cav-1 overexpression with drug resistance are congruent with data associating cav-1 upregulation with androgen resistance and survival. These observations suggest a possible overlapping, common protective/survival function for cav-1 in regard to androgen and drug resistance.

Cav-1 is Secreted by Prostate Cancer Cells and Secreted Cav-1 has Biological Activities

It was of interest that even in patients with hormone refractory metastases, who had the highest levels of cav-1 positive cells in their tumors (both frequency and % positive cells) that the percentage of cav-1 positive cell did not exceed 40%. These data indicate that cav-1 functions as a paracrine/autocrine factor. This prospect together with a recent report that cav-1 is secreted by pancreatic acinar cells led to an investigation of whether prostate cancer cells also secrete cav-1.

Cav-1 was detected in conditioned media from androgen-insensitive mouse and human (DU145, PC3 and TSU-Pr1) prostate cancer cells in variable amounts. In androgen-sensitive, low passage LNCaP cells (LP-LNCaP) cav-1 was not expressed.

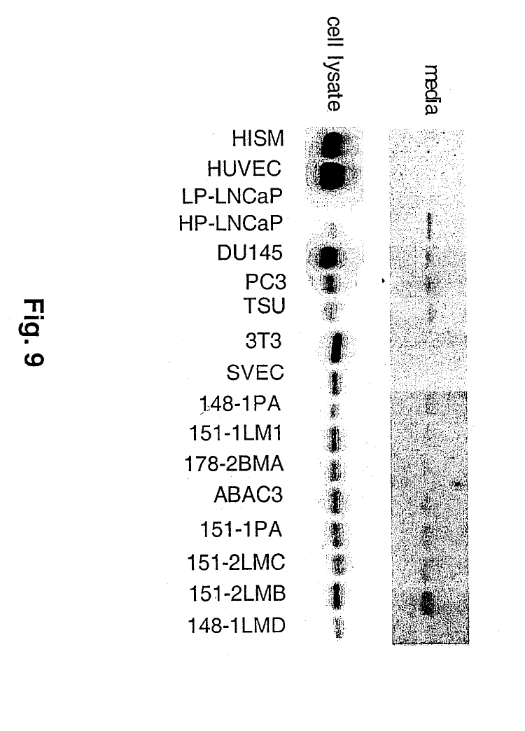

However, in high passage LNCaP cells (HP-LNCaP) that had reduced androgen-sensitivity, cav-1 was expressed and secreted into the medium. In contrast, non-prostatic cells, such as endothelial, fibroblast, and smooth muscle, had a substantial amount of intracellular cav-1 yet minimal or nondetectable levels of cav-1 in their conditioned media (FIG. 9).

Mouse prostate cancer cell line, 178-2BMA, derived from a bone metastasis generated from the metastatic mouse prostate reconstitution model and HP-LNCaP were used to test the possible regulation of cav-1 secretion by dihydrotestesterone (DHT) and Dex in vitro. Both cell lines were shown to be insensitive to androgen in vitro, i.e., no significant changes in cell number or viability were detected under serum free conditions in the presence or absence of 10 nM T. However, cav-1 was secreted by 178-2BMA and LNCaP cells in response to these steroid hormones. This increase in secreted cav-1 in response to these secretagogues was paralleled by a decrease in intracellular cav-1. The secretory route for cav-1 by expressing human cav-1 in cav-1 negative LP-LNCaP cells was also investigated. Following transfection, a substantially greater amount of ectopically expressed cav-1 was detected in the media than in the cell lysate. Further cav-1 secretion was increased in response to DHT. Cav-1 was not detected in the media or cell lysate of the vector control transfected cells, yet all transfected cells excreted prostate specific antigen (PSA) into the media in a DHT-regulated fashion. Ectopically expressed cav-1 is secreted by LNCaP cells and the secreted cav-1 migrates on SDS-PAGE similarly to that derived from endothelial cells and fibroblasts, suggesting that the secreted form is not modified post-transcriptionally.

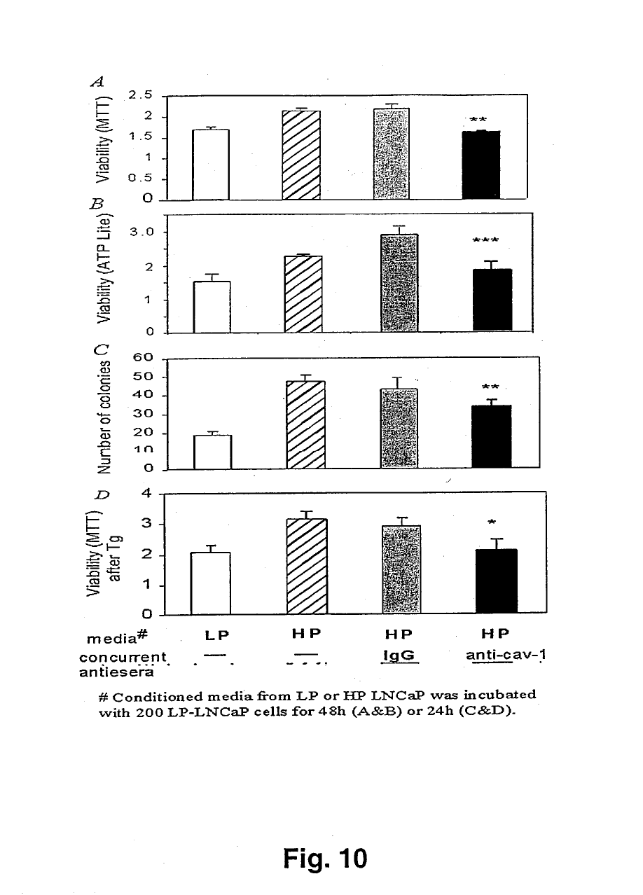

The functional activity of secreted cav-1 (concentrated conditioned media collected from HP-LNCaP cells) was investigated by testing the effects on LP-LNCaP cell viability and clonal growth under serum-free conditions. The results indicate that secreted cav-1 was capable of promoting viability, using a standard MTT method (FIG. 10A) or luminescent technique (Packard ATPLite) (FIG. 10B), and of stimulating viability/clonal growth using a clonogenic assay (FIG. 10C). To test whether such activities would be specific for the cav-1 molecule, polyclonal cav-1 antibody was added to conditioned media or rabbit IgG as a control. Treatment of the conditioned media with anti cav-1 antibody reduced the viability significantly (P<0.001 for MTT and clonogenic assays and P<0.0001 for ATP Lite assay) compared to the IgG-treated medium. It was also found that secreted cav-1 was able to protect LP-LNCaP cells from thapsigargin (Tg) induced apoptosis (FIG. 10D). These studies revealed that media containing secreted cav-1 generates anti-apoptotic activities in prostate cancer cells similar to those elicited following enforced expression of cav-1 within the cell.

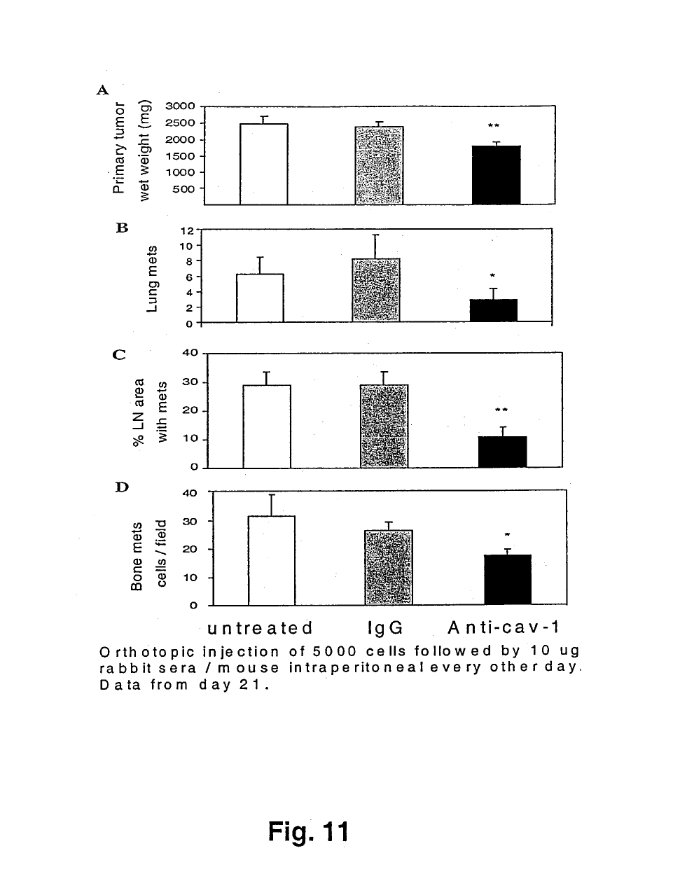

It was then tested whether blocking secreted cav-1 activity in vivo with specific antibodies potentially could result in therapeutic activity through abrogation of the anti-apoptotic effects of secreted cav-1. Androgen-insensitive 178-2BMA cells that spontaneously metastasize with high frequency (nearly 100%) to lung, lymph nodes and bone were grown as orthotopic tumors in adult male mice. After 21 days of treatment with cav-1 antibody or IgG, the animals were sacrificed. The mean tumor wet weight (FIG. 11A) and the mean number of lung metastases (FIG. 11B) of the anti-cav-1 treated group were significantly lower than those in the IgG-treated group (P<0.01 and P<0.05, respectively). The cav-1 antibody treated group also had a significantly lower percentage of cancer cell volume in lymph nodes (P<0.01) (FIG. 11C). The metastatic cell density in the bone marrow (FIG. 11D) was also reduced significantly (P<0.05) in the cav-1 antibody-treated mice than in those of the IgG-treated group. These results show that neutralization of secreted cav-1 in vivo by specific antibody suppresses primary prostate tumor growth and spontaneous metastasis to the lung, lymph nodes, and bone.

Serum Cav-1 is a Potential Bio-marker for Prostate Cancer

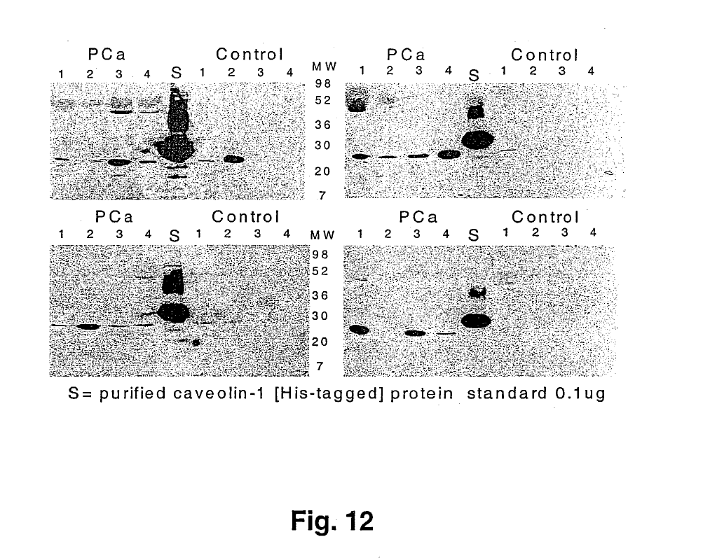

For the studies described in the above sections, immunohistochemical analysis of prostate specimens, such as those obtained at the time of surgery, were relied on to analyze cav-1 expression. Because cav-1 was secreted by prostate cancer cells, human serum from men with (n=32) or without (n=32) prostate cancer was evaluated. Serum samples were fractionated by ultracentrifugation and analyzed distinct lipid fractions for cav-1 by western blotting. As depicted in FIG. 12, cav-1 was detected in the HDL₃ fraction of human serum at higher levels in prostate cancer patients with metastatic disease (positive lymph nodes) than in serum obtained from men without prostate cancer. As demonstrated in this series of 4 western blots, 14 of 16 men with prostate cancer had detectable serum cav-1, whereas only 4 of 16 age-matched men without prostate cancer had a signal. Similar data were obtained for an additional 16 patients and 16 control serums. Cav-1 migrates with an apparent molecular weight of 22 kD, and larger aggregates typically are seen. The standard of recombinant purified cav-1 has a 6X-His tag and migrates somewhat more slowly than the authentic cav-1.

These observations are consistent with the view that HDL₃ is the principal acceptor of excess cholesterol in the cell's plasma membrane, and that cav-1 plays a significant role in transporting cholesterol to that site. Caveolae are a major site of cholesterol efflux from cells, and cav-1 mediates the intracellular movement of cholesterol; in prostate cancer cells, cav-1 may also be transported out of the cell. The expression of cav-1 mRNA is regulated by cholesterol and the level of low density lipoprotein. A recent report has defined a subset of breast cancer patients with cav-1 mutations at codon 132. Also mutations in the muscle-specific cav-3 gene have been associated with autosomal diseases. In studies of cav-1 in prostate cancer, no specific mutations in cell lines or selected pathologic specimens were detected. However, it is conceivable that mutations of cav-1 may play a role in abnormalities of cholesterol metabolism.

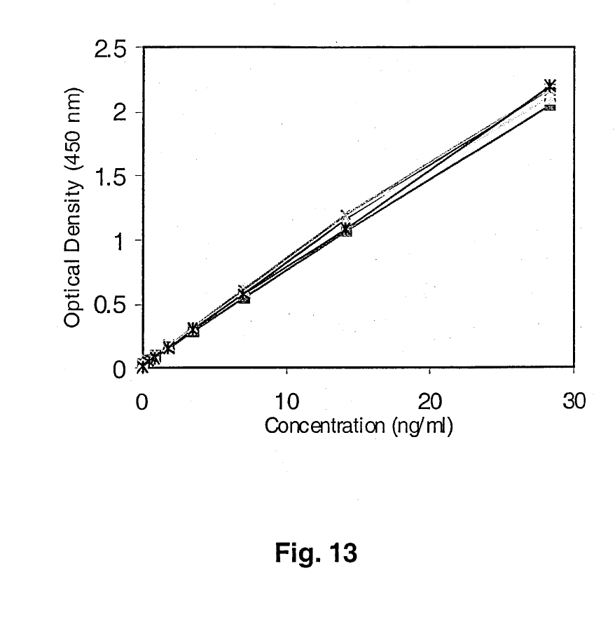

Since western blotting analysis is semi-quantitative, a more quantitative an ELISA analysis of serum cav-1 was developed for the analysis of cav-1. Using a purified recombinant cav-1 protein as a standard, a quantitative direct sandwich ELISA was optimized and demonstrated its reproducibility (FIG. 13). This figure depicts the superposition of five standard curves as measured at 5-day intervals with the same lot of reagents. It is apparent that all five standard curves are similar regarding sensitivity and working range of the assay. The high reproducibility of the assay is reflected by the low intra-and inter-assay variances. Future modifications of this assay may result in improvements (e.g., sensitivity). However, this first generation cav-1 ELISA is a validated tool that is capable of accurately performing analyses.

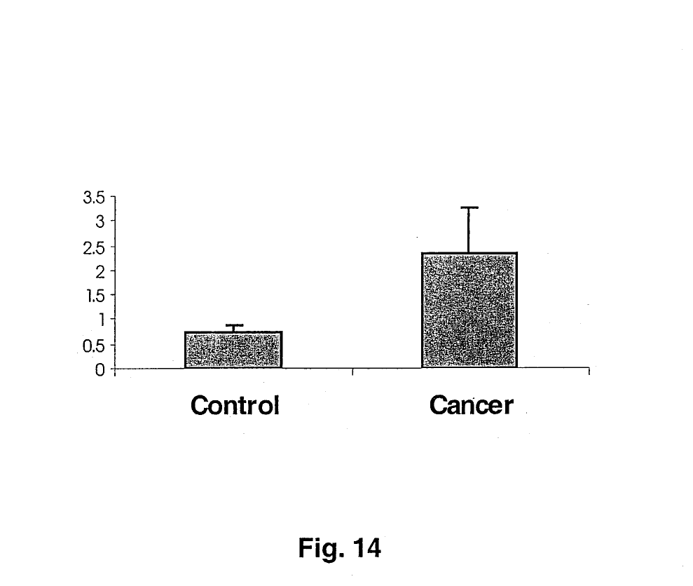

To determine the potential for serum cav-1 to be useful clinically, serum cav-1 was analyzed in two preliminary studies. In the first study an analysis of serum cav-1 levels in 117 men with clinically localized lymph node negative prostate cancer, with a median serum PSA level of 5.8 ng/ml was compared to a group of 115 control men with serum PSA levels that have been under 1.5 ng/ml over a two-year time period (Table 1 and FIG. 14). The pre-operative (pre-op) serum cav-1 levels were significantly higher in the prostate cancer group (P=0.027, Mann-Whitney test).

TABLE 1
Serum samples for cav-1 ELISA

| | | | Lower 5th |
|---|---|---|---|
| | Control | Prostate Cancer | percentile of |
| | Mean ± st.dev. | Mean ± st.dev. | Prostate Cancer |
| N | 115 | 117 | Patients |
| Age (Yr) | 57.9 ± 8.4 | 60.9 ± 6.3 | |
| PSA (ng/ml) | 0.63 ± 0.29 | 7.45 ± 6.12 | 8.6% (10/116) |
| Cav-1 (ng/ml) | 0.73 ± 1.63 | 2.35 ± 9.83 | 93.2% (109/117) |

In this study, correlation analyses of cav-1 serum levels with specific clinical and pathological parameters associated with prostate cancer specimens including (Gleason grade, positive margin status extracapsular extension and seminal vesicle invasion) failed to demonstrate a significant relationship. This could indicate either an insufficient or inappropriate set of prostate cancer specimens or that cav-1 serum levels reflect a completely unique parameter of aggressive virulent disease. Unlike serum PSA, which can show considerable variability within the range of 2-10 ng/ml, cav-1, because of its close linkage with the biology of the disease, may, within certain limits of detection, be more definitive in regard to its prognostic potential. The distribution of serum cav-1 levels in the prostate cancer patient serums in this group were examined. For comparison, also analyzed was the distribution of serum PSA levels (see Table 1). The results indicated that serum cav-1 levels were not normally distributed (skewness=8.1, kurtosis=73.0) and generated a consistently low “background” value with most patients having levels at or near the limit of detection. On the other hand, serum PSA levels were less skewed (skewness=3.5, kurtosis=18.5) than those of serum cav-1. The comparative distribution profiles suggest that cav-1 may give clearer negative-positive information as indicated by the observed fact that 93.2% of patients had serum cav-1 levels in the lowest fifth percentile of the distribution curve (<4.79 ng/ml) and 8.6% of the patients' serum PSA levels lying in the lower fifth percentile (<2.91 ng/ml) (see Table 1).

In the second preliminary study the post-radical prostatectomy serum cav-1 samples from another group of 55 patients with advanced prostate cancer and a median follow-up time of 36 months after surgery was analyzed. Preoperative serum specimens were not available for these patients. Post-operative serum cav-1 correlated significantly with Gleason grade 1 (i.e., predominant Gleason grade in the specimen) (P=0.025 Spearman Rank Correlation), seminal vesicle invasion (P=0.030, Spearman Rank Correlation) and lymph node involvement (P=0.023, Spearman Rank Correlation) (Table 2).

TABLE 2
Correlation of post-operative serum cav-1 levels with
biochemical and pathological parameters in 55 patients
post-radical prostatectomy

| | | Cav-1 significance |
|---|---|---|
| | Median (range) | P value |
| | [frequency] | (Spearman Rank) |
| Age | 62 (46-73) | 0.053 |
| Cav-1 Post-Op | 0.3 (0-48.4) | |
| (ng/ml) | | |
| PSA Pre-Op (ng/ml) | 6.8 (1.7- | 0.098 |
| | 31.0) | |
| Gleason Grade (1) | 3 (2-4) | 0.025 (r = 0.31) |
| Gleason Grade (2) | 3 (3-5) | 0.871 |
| Gleason Grade | 7 (5-9) | 0.08 |
| (Total) | | |
| Positive margins | 13.2% [7/53] | 0.363 |
| Extracapsular | 62.3% [33/53] | 0.300 |

The serum cav-1 association with time to disease recurrence in this group of 55 patients was determined using Cox proportional hazard regression analysis (Table 3). Post-operative serum cav-1 is a significant univariate predictor for shorter time to biochemical recurrence after surgery (P=0.005). Significant univariate prediction value for disease progression was also found for several pathological parameters such as Gleason grade (P=0.010), Gleason Score (P=0.004), preoperative PSA level (P=0.025, normalized via natural log transformation), seminal vesicle invasion (P=0.003), extracapsular extension (P=0.019), and positive lymph nodes (P=0.006). Overall these data indicates that postoperative serum cav-1 levels may provide unique information regarding the biological and clinical potential of prostate cancer in specific patient populations.

TABLE 3
Univariate analysis of serum cav-1 and biochemical and
pathological parameters to predict time to recurrence.

| | | Risk | | |
|---|---|---|---|---|
| Parameters | N | Ratio | 95% confidence interval | P value |
| Serum cav-1 (Post-Op) | 53 | 1.073 | 1.021-1.127 | 0.005 |
| Serum PSA* (Pre-Op) | 51 | 2.472 | 1.122-5.442 | 0.169 |
| Gleason Grade (1) | 51 | 3.922 | 1.396-11.0022 | 0.01 |
| Gleason Grade (2) | 51 | 1.714 | 0.678-4.335 | 0.255 |
| Gleason Grade (Total) | 51 | 2.663 | 1.368-5.816 | 0.004 |
| Positive margins | 51 | 2.819 | 0.908-8.754 | 0.073 |
| Extracapsular extension | 51 | 5.866 | 1.330-25.869 | 0.019 |
| Seminal vesicle invasion | 52 | 4.588 | 1.662-12.668 | 0.003 |
| Lymph Node Positive | 52 | 8.159 | 1.853-35.926 | 0.006 |

Other embodiments are uses of the invention will be apparent to those skilled in the art from consideration of the specification and practice of the invention disclosed herein. All documents cited herein for whatever reason, including U.S. Provisional Application No. 60/352,513, are specifically and entirely incorporated by reference. The specification including the examples should be considered exemplary only, are the true scope of the invention defined by the following claims.

1. A method for detecting a neoplasia in a patient comprising determining a level of a caveolin in a biological sample obtained from the patient.

2. The method of claim 1 where the neoplasia is selected from the group consisting of breast cancer, esophageal cancer, head and neck cancer, liver cancer, lung cancer, gastrointestinal cancer, pancreatic cancer, prostate cancer, skin cancer, stomach cancer, a metastasis, a micrometastasis, and combinations thereof.

3. The method of claim 2 wherein the cancer is a micrometastasis of prostate cancer.

4. The method of claim 1 wherein the patient is a mammal.

5. The method of claim 1 wherein the caveolin is selected from the group consisting of caveolin-1, caveolin-2, caveolin-3 and fragments and combinations thereof.

6. The method of claim 1 wherein the caveolin is caveolin-1.

7. The method of claim 1 wherein the caveolin is contained within a lipoparticle.

8. The method of claim 1 wherein the biological sample is selected from the group consisting of blood, plasma, serum, tissue, interstitial fluid, spinal fluid, and combinations thereof.

9. The method of claim 1 wherein the level of caveolin determined is compared to known levels of caveolin that were determined from similar biological samples obtained from normal patients.

10. The method of claim 1 wherein the level of the caveolin is determined from two or more biological samples obtained from the same patient, and the difference in caveolin levels found between the biological samples compared to detect said neoplasia in the patient.

11. A kit for detecting the level of a caveolin in a biological sample obtained from a patient suspected of having a neoplastic disorder comprising an agent that can be quantitatively detected upon association with said caveolin.

12. The kit of claim 11 wherein the agent is selected from the group consisting of an anti-caveolin antibody, a caveolin receptor, and functional fragments and combinations thereof.

13. The kit of claim 11 wherein the agent is detectable by chromatography, electrical capacitance, fluorescence, luminescence, mass, molecular weight, radioactivity, or a combination thereof.

14. A method for detecting prostate cancer in a patient comprising:
obtaining a sample of blood from a patient;
fractionating said sample into one or more fractions;
determining a level of caveolin in at least one fraction;
comparing the level of caveolin determined in said fraction with the level of caveolin determined in fractions obtained from similar biological samples obtained from non-cancerous patients; and
determining the presence or absence of prostate cancer in said patient.

15. The method of claim 14 wherein the prostate cancer is metastatic prostate cancer.

16. The method of claim 14 wherein the non-cancerous patients are patients with benign prostatic hyperplasia.

17. A method for determining a metastatic potential of a primary prostate tumor comprising:
coupling an anti-caveolin antibody to a detectable marker contacting a sample of the tumor with the anti-caveolin antibody coupled to the detectable marker; and
determining the amount of anti-caveolin antibody bound to the sample.

18. The method of claim 17 wherein the detectable marker is selected from the group consisting of fluorescent markers, luminescent markers, radioactive markers, visible markers, and combinations thereof.

19. A method for determining a metastatic potential of a primary prostate tumor comprising:
coupling an anti-caveolin antibody to a detectable marker
contacting a sample of the tumor with the anti-caveolin antibody coupled to the detectable marker; and
determining the amount of anti-caveolin antibody bound to the sample.
The method of claim 1 wherein the anti-caveolin antibody is a monoclonal or polyclonal antibody.

20. The method of claim 19 wherein the anti-caveolin antibody is coupled to a detectable label.

21. A reagent for determining the metastatic potential of a primary prostate tumor comprising an anti-caveolin antibody coupled to a detectable marker.

22. The reagent of claim 21 wherein the anti-caveolin antibody is monoclonal or polyclonal.

23. The reagent of claim 21 wherein the anti-caveolin antibody is coupled to a detectable label.

24. A method for detecting prostate cancer, comprising:
extracting a serum sample from a patient;
separating the serum sample into lipid fractions;
contacting the HDL₃ fraction of serum sample with an anti-caveolin antibody coupled to a detectable marker;
measuring the amount of anti-caveolin antibody bound to the HDL₃ fraction of the serum sample; and
determining the caveolin-1 concentration in the serum sample.

25. The method of claim 24 wherein the anti-caveolin antibody is monoclonal or polyclonal.

26. The method of claim 24 wherein the anti-caveolin antibody is coupled to a detectable label.
